(19) United States
(12) Patent Application Publication
(10) Pub. No.: US 2024/0225988 A1
(43) Pub. Date: Jul. 11, 2024

(54) FLUID COSMETIC COMPOSITIONS WITH UNUSUAL RHEOLOGIC CHARACTERISTICS, POLYMERS ADAPTED TO PROVIDE THE ABOVE SAID CHARACTERISTICS AND THEIR SYNTHESIS

(71) Applicant: INTERCOS S.p.A., MILANO (IT)

(72) Inventors: Patrizia VALSESIA, CALCO LC (IT); Gaetano DISTEFANO, BERGAMO (IT); Nella GALOTTO, LOMAGNA LC (IT); Claudio PIROVANO, VERDERIO LC (IT)

(73) Assignee: INTERCOS S.p.A., MILANO (IT)

(21) Appl. No.: 18/563,595

(22) PCT Filed: May 24, 2022
(86) PCT No.: PCT/IB2022/054827

§ 371 Date: Nov. 22, 2023

(30) Foreign Application Priority Data
May 25, 2021 (IT) 102021000013463

(51) Int. Cl. A61K 8/72; A61Q 1/06

(52) U.S. Cl. CPC A61K 8/72; A61Q 1/06; A61K 2800/20; A61K 2800/54

(57) ABSTRACT

A fluid cosmetic composition with unusual rheologic characteristics has a formulation comprising 0.1 to 50% by weight of at least one fluid branched polymer obtained by simultaneous reaction from components A. B and C, where A is a linear polymer with a molecular weight Mw between 5000 and 50000 Da, provided with reactive functional groups X in a chain at a concentration between 0.1 and 50 mmol/g of polymer. B is a linear polymer with a molecular weight Mw between 10000 and 100000 Da, having terminal reactive functional groups of type Y capable of reacting with the functional group X in an addition or condensation reaction, and C is a molecule or linear oligomer (or a mixture thereof) with a molecular weight between 50 and 1000 Da, having a reactive functional group Y per molecule, capable of reacting with the functional group X in an addition or condensation reaction. The molar ratio of the functional groups of B and A is between 0.01 and 0.5 and the molar ratio of the functional groups of C and A is between 0.5 and 1.2. The reactive group X of A is capable of reacting with the reactive sites Y of B and C.

The present invention relates to a series of fluid cosmetic compositions, more precisely non-solid cosmetic compositions with the rheologic characteristics of viscoelastic fluids, which are suitable for creating corresponding fluid cosmetic products with unusual rheologic characteristics adapted to determine extended application times, considerable filming character, long duration, and non-transferability.

The invention also relates to polymers usable for giving cosmetic compositions such unusual rheologic characteristics, as well as to the synthesis thereof.

The continuous need in cosmetics to create sophisticated products, with refined finishings, unexpected sensory experiences and advanced functionalities pushes towards the design and conception of new cosmetic raw materials which can be decisive for the creation of the aforesaid effects, and in particular for obtaining the desired sensory transformation in the final cosmetic product.

In particular, the invention described below is directed to obtaining cosmetic products having an unusual texture as compared to those traditionally found in similar products on the market, characterized by the ability to generate, during the application to the user's body, a force perpendicular to the flowing direction (here referred to as the Normal Force or F_{N}). Such a force opposes the thinning of the formulation between the applicator and the application surface (for example, between the fingers and the face skin as in the case of a foundation) giving the formulation appreciated distinctive characteristics.

In order to better understand this concept, consider that there are naturally occurring lubricating fluids generated from aqueous biopolymer solutions. Such biopolymers, synthesized by the tissues, allow giving consistency and body to secretions: for example, mucins are glycoproteins present in the mucous secretions of the respiratory and gastro-intestinal tract, with a lubricating and protective function. The primary characteristic of such physiological fluids is the viscosity and the property of being “stringy”. From a rheologic point of view, mucus has the so-called Weissenberg effect according to which a fluid, when rotated by means of an impeller, climbs on the rod (“rod climbing” effect) of the impeller itself. This behavior is quite common in the case of high-branched hydrocolloid solutions, such as concentrated solutions of xanthan gum, which are slimy, elastic, stringy, and show the Weissenberg effect. The filming and lubricating effect is obtained by virtue of the fact that such fluids, when placed under the action of a direct shear force along a direction (for example between two surfaces in relative motion), generate a force perpendicular (Normal Force, F_{N}) to such a direction which separates the two surfaces in relative motion.

Fat-soluble polymers with the characteristics of the above-mentioned hydrocolloids are uncommon, while silicone fluids are widely used in cosmetic compositions, as shown for example by EP 1010715 A1, providing the use of an alkyl-silicone elastomer, and by US 2012/156148 A1, providing the use of a silicone reagent (e.g., organopolysiloxane) in the presence of a fluid carrier strongly characterizing the properties of the material. Depending on the molecular weight and crosslinking, different cosmetic performances and applications can be obtained.

Consider now that a cosmetic composition with lubricating fluids is subjected to a controlled flow, for example interposed between two parallel flat surfaces placed in relative rotary motion. Due to such a rotary motion, the fluid cosmetic composition generates a force of mutual separation of the two surfaces, which is perpendicular to the surfaces themselves and is expressed in the form of pressure Pa (N/mm²), dividing F_{N} (expressed in Newton or N) by the area (expressed in mm²) of the material being flowed.

The generated F_{N} is proportional to the shear rate and shows an increasing trend until reaching a maximum value (F_{N,MAX}) beyond which F_{N} decreases.

The shear rates to be considered are those in the range between 0.1 s⁻¹ and 1000 s⁻¹, in which the shear rates of the topical application of a cosmetic product fall.

Starting from this principle, a fluid cosmetic composition has been obtained with the present invention, which has a lubricated sensory nature as a function of the fact that, by opposing a contrary force to that applied in use perpendicularly to the user's body, the composition allows maintaining a thickness of material between the surfaces under relative motion (for example, hands/face or applicator/lips) during the application.

The application time or “playtime”, during which the cosmetic product is applied and can be shaped, is highly extended, a melting perception (ability of the product to melt on the skin and become uniform thereon) is caused and does not fade thus making the cosmetic product highly adaptable to the skin. Furthermore, the elasticity of the fluid composition used gives the product a considerable filming character but disconnected from uncomfortable sensations such as dryness and rigidity due to the drying of the most commonly used film-forming agents. The creation of a homogeneous and comfortable film is reflected in highly desirable properties such as the long duration (“long lasting”) and non-transferability (“no transfer”) of the formulations.

This behavior was obtained by virtue of the introduction of fluid branched polymers having strong elastic and lubricating properties into the formula.

More precisely, in accordance with the present invention, a fluid cosmetic composition was obtained, which unlike the teachings of EP 1010715 A1 and US 2012/156148 A1, is characterized in that it has a formulation comprising 0.1 to 50% by weight of at least one fluid branched polymer obtained by reaction of the following components A, B and C:
A) linear polymer with a molecular weight Mw between 5000 Da and 50000 Da (expressed in equivalents of polystyrene), provided with reactive functional groups X in a chain at a concentration between 0.1 and 50 mmol/g of polymer;
B) linear polymer with a molecular weight Mw between 10000 Da and 100000 Da (expressed in equivalents of polystyrene), having terminal reactive functional groups of type Y capable of reacting with the functional group X in an addition or condensation reaction;
C) molecule o linear oligomer with a molecular weight between 50 Da and 1000 Da (or a mixture thereof) having a reactive functional group Y per molecule, capable of reacting with the functional group X in an addition or condensation reaction;
   wherein
the molar ratio of the functional groups of B and A is between 0.01 and 0.5 (mol Y_{B}/mol X_{A}),
the molar ratio of the functional groups of C and A is between 0.5 and 1.2 (mol Y_{C}/mol X_{A}), and
the reactive group X of A is capable of reacting with the reactive sites Y of B and C.

The aforesaid fluid branched polymer has the characteristics of a viscoelastic fluid with shear thinning behavior and is capable of generating, when rotated between two flat plates 1 mm apart and with a 50 mm diameter, for shear rates between 0.1 s⁻¹ and 1000 s⁻¹, at a temperature of 20° C., a Maximum Normal Force (F_{N, MAX}) of at least 6 N, corresponding to a Maximum Normal Pressure (P_{N,MAX}) of 3000 Pa.

Such branched polymers appear as viscous fluids at temperatures between 10° C. and 70° C. and, when subjected to a shear force, the viscosity thereof decreases (“shear thinning”): therefore, they are materials that, while they are spread, reduce the viscosity thereof in a direction parallel to the flow and simultaneously generate a “cushion” effect in a direction perpendicular to the flow, with consequent unique lubricating characteristics.

The polymers A and B can be selected from the major families of polymers used in cosmetics such as, by way of non-limiting example, silicones, polysaccharides, polyesters, polyethers, and polyurethanes.

The reactive functional groups X and Y can be conveniently selected from, but not limited to, complementary reactive groups such as Si—H, carboxyl, vinyl, hydroxyl, alkoxy (—O—CH₃, —O—CH₂CH₃, —O—CH(CH₃)₂) groups, amines, isocyanates.

A class of such fluid branched polymers is represented by branched alkyl dimethicones in which:
A) is preferably selected from polymethylhydrosiloxanes with the structural formula:

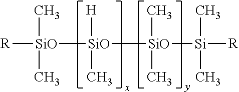

with x>2; R═H, CH₃
B) is preferably selected from linear polydimethylsiloxanes with terminal vinyl groups according to the structural formula:

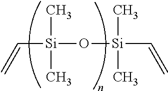

wherein n identifies a polymer with a molecular weight Mw between 10000 Da and 100000 Da
C) is preferably selected from alpha olefins of general formula CH₂═CH—R, with R being an alkyl group containing from 6 to 50 carbon atoms.

Such a branched polymer can be advantageously synthesized through the hydrosilylation reaction in which polymethylhydrosiloxane A) is reacted simultaneously with a polydimethylsiloxane terminated with vinyl groups B) and with an alpha olefin C), in the presence of a hydrosilylation catalyst (Karstedt catalyst).

Another class of such fluid branched polymers is represented by branched cellulose derivatives in which:
A) is preferably selected from cellulose derivatives, in particular ethyl cellulose, selectable from the commercial ethyl cellulose grades ensuring the presence of the appropriate number of hydroxyl groups in the structure.
B) is preferably selected from linear polyesters with terminal isocyanate groups.
C) is preferably selected from an aliphatic mono-isocyanate of general formula O═C═N—R, with R being an alkyl group containing from 6 to 50 carbon atoms.

Such a fluid branched polymer can be advantageously synthesized through the reaction in which the hydroxyls of ethyl cellulose A) are reacted simultaneously with a linear polyester with terminal isocyanate groups B) and with aliphatic mono-isocyanate C), in the presence of a Zn-based catalyst.

A further class of such fluid branched polymers is represented by branched polyester derivatives in which:
A) is preferably selected from polyglycerols or from polyglycerol esters, selectable from those ensuring the presence of the appropriate number of hydroxyl groups in the structure.
B) is preferably selected from the linear polyesters terminated with an acid group.
C) is preferably selected from an aliphatic mono-acid, having an alkyl group containing from 6 to 50 carbon atoms.

Such a branched polymer can be advantageously synthesized through the esterification reaction in which a polyglycerol A) is reacted simultaneously with a linear polyester di-acid B) and with an aliphatic mono-acid C), in the presence of an Sn-based catalyst.

The fluid branched polymers of the present invention were unexpectedly obtained by synthesis in a single step between A, B and C. This contrivance in the synthesis step and the careful choice of the molar ratio of the reactive groups of A, B and C are crucial aspects for the formation of a fluid branched polymer with the rheologic characteristics described above.

The use of raw materials with the described rheologic behavior, such as those according to the present invention, in a formulation gives the formulation itself the particular properties not obtainable with the use of the traditional raw materials. In particular, the formulations thus obtained have a long application time or “playtime” and optimal wear resistance (high “long-wear” and “long-lasting”). The rheologic characteristic of the raw material is inherited from the various fluid formulations described in the present invention.

Some formulation examples of fluid cosmetic compositions with unique rheologic properties according to the present invention are shown below.

The cosmetic products of the invention, such as lip fluid, foundation, concealer and primer (examples 1, 2, 3, 4, 5, 6 and 7), have very particular textures due to the advanced rheologic properties of the materials contained therein.

Such rheologic characteristics make the cosmetic compositions very special. The lip fluid (example 1) is very comfortable when applied and over time with a “no-transfer” effect since the material (Sample II) creates a soft but wear-resistant thin film. The same rheologic characteristics are maintained by the formulation with Sample V of a non-silicone lip fluid (example 6). Unlike the usual “no-transfer” cosmetic compositions, those discussed, when applied, do not dry the lips and do not create an annoying tightening effect. The fluid cosmetic compositions for the face (examples 2, 3, 4, 5 and 7), by virtue of the presence of the raw materials discussed (Sample I, Sample II, Sample III, Sample IV and Sample VI), are very comfortable when applied and over time, giving a “no-transfer” effect and softness.

### Raw Materials Used

Sample I: very elastic fluid with unique properties, which gives a unique sensory effect when applied with a long playtime. Due to the elastic and soothing nature thereof, the cosmetic composition is very comfortable, soft and adherent to the skin for a long time.

| | | %(w/w) |
|---|---|---|
| PHASE A | Polymethylhydrosiloxane | 38.95 |
| PHASE B | Vinyl polydimethylsiloxane | 26.0 |
| PHASE C | Hexadecene | 20.0 |
| PHASE D | Hydrosilylation catalyst | 0.05 |
| PHASE E | Alkyl silicone solvent | 15.0 |

Sample II: elastic fluid with a unique and very controlled sensory profile despite its fluidity. Being very adherent, it creates a continuous thin film being comfortable over time.

| | | %(w/w) |
|---|---|---|
| PHASE A | Polymethylhydrosiloxane | 44.95 |
| PHASE B | Vinyl polydimethylsiloxane | 28.0 |
| PHASE C | Hexadecene | 17.0 |
| PHASE D | Hydrosilylation catalyst | 0.05 |
| PHASE E | Dimethicone solvent | 10.0 |

Sample III: branched alkyl silicone with high molecular weight. The silicone nature gives the material softness, while the waxy (alkyl) nature makes the material creamy and melting when applied. It can be used in all product categories, giving a pleasant creaminess.

| | | %(w/w) |
|---|---|---|
| PHASE A | Polymethylhydrosiloxane | 47.95 |
| PHASE B | Vinyl polydimethylsiloxane | 28.0 |
| PHASE C | Olefin C20-24 | 26.0 |
| PHASE D | Hydrosilylation catalyst | 0.05 |

Sample IV: fluid raw material (60° C.). Partially cross-linked alkyl silicone functionalized with waxy chains. It has the ability to modify the rheology of medium/high viscosity silicone oils and fluid alkyl silicones. The viscosized cosmetic oil is elastic and when applied gives shine and creaminess.

| | | %(w/w) |
|---|---|---|
| PHASE A | Polymethylhydrosiloxane | 47.95 |
| PHASE B | Vinyl polydimethylsiloxane | 28.0 |
| PHASE C | Olefin C24-28 | 26.0 |
| PHASE D | Hydrosilylation catalyst | 0.05 |

Sample V: branched ethyl cellulose made fluid-elastic at temperatures below 60° C. by virtue of the branching and alkyl functionalization of the solid raw material at room temperature. It has the ability to modify the rheology of low-polarity oils and esters.

| | | %(w/w) |
|---|---|---|
| PHASE A | Ethyl cellulose | 49.95 |
| PHASE B | Isocyanate-terminated polyester prepolymer | 27.0 |
| PHASE C | Octadecyl isocyanate | 23.0 |
| PHASE D | Zn-based catalyst | 0.05 |

Sample VI: partially cross-linked polyester functionalized with alkyl chains. It has the ability to modify the rheology of medium-high polarity oils and esters. The cosmetic oil is elastic and when applied gives shine and creaminess

| | | %(w/w) |
|---|---|---|
| PHASE A | Polyglycerol | 45.95 |
| PHASE B | Polyester di-acid | 26.0 |
| PHASE C | Lauric acid | 28.0 |
| PHASE D | Sn-based catalyst | 0.05 |

### EXAMPLES

### Example 1: Lip Fluid

| | | %(w/w) |
|---|---|---|
| PHASE A | Sample II | 8.0 |
| | Skin conditioners | 21.0 |
| | Antifoam agents | 18.0 |
| PHASE B | Soothing agent | 4.0 |
| | Viscosity enhancers | 12.0 |
| PHASE C | Solvent | 18.0 |
| | Dispersants | 1.3 |
| | Skin conditioners | 3.2 |
| | Dyes | 14.5 |

### Example 2: Foundation

| | | %(w/w) |
|---|---|---|
| PHASE A | Skin conditioners | 19.0 |
| | Emulsifiers | 9.0 |
| | Viscosity enhancers | 10.0 |
| PHASE B | Sample I | 4.0 |
| | Sample III | 2.0 |
| PHASE C | Dyes | 18.3 |
| PHASE D | Preservatives | 1.0 |
| PHASE E | Solvents | 33.0 |
| | Viscosity enhancers | 2.0 |
| | Chelating agents | 0.2 |
| PHASE F | Antioxidants | 0.5 |
| | Fragrances | 1.0 |

### Example 3: Concealer

| | | %(w/w) |
|---|---|---|
| PHASE A | Skin conditioners | 14.0 |
| | Emulsifiers | 3.0 |
| | Surfactants-emulsifiers | 2.0 |
| PHASE B | Volumizers | 0.8 |
| PHASE C | Sample I | 2.5 |
| PHASE D | Solvents | 12.5 |
| | Dispersants | 1.2 |
| | Emulsifiers | 0.5 |
| | Dyes | 28.0 |
| PHASE E | Dyes | 1.5 |
| | Surface modifiers | 1.0 |
| PHASE F | Preservatives | 1.0 |
| PHASE G | Solvents | 29.3 |
| | Viscosity enhancers | 2.0 |
| | Chelating agents | 0.2 |
| PHASE H | Antioxidants | 0.5 |

### Example 4: Foundation

| | | %(w/w) |
|---|---|---|
| PHASE A | Skin conditioners | 2.0 |
| | Anti-caking agent | 0.5 |
| PHASE B | Skin conditioners | 6.0 |
| | Sample II | 2.0 |
| | Emulsifiers | 3.5 |
| | Antioxidants | 0.5 |
| | Antifoam agents | 1.0 |
| PHASE C | Solvents | 4.0 |
| | Dispersants | 1.0 |
| | Emulsifiers | 0.5 |
| | Skin conditioners | 2.5 |
| | Soothing agent | 2.3 |
| | Volumizers | 1.0 |
| | Dyes | 25.0 |
| PHASE D | Solvents | 28.0 |
| | Viscosity enhancers | 4.0 |
| | Chelating agents | 0.5 |
| PHASE E | Film-forming agent | 7.0 |
| PHASE F | Preservatives | 1.0 |
| PHASE G | Anti-caking agent | 2.0 |
| | Binder | 2.5 |
| | Dyes | 2.5 |
| PHASE H | Fragrance | 0.7 |

### Example 5: Primer

| | | %(w/w) |
|---|---|---|
| PHASE A | Sample IV | 35.0 |
| PHASE B | Skin conditioners | 25.0 |
| | Soothing agent Sample II | 40.0 |

### Example 6: Non-Silicone Lip Fluid

| | | %(w/w) |
|---|---|---|
| PHASE A | Sample V | 9.0 |
| | Skin conditioners | 20.0 |
| | Antifoam agents | 18.0 |
| PHASE B | Soothing agent | 6.0 |
| | Viscosity enhancers | 10.0 |
| PHASE C | Solvent | 18.0 |
| | Dispersants | 1.3 |
| | Skin conditioners | 3.2 |
| | Dyes | 14.5 |

### Example 7: Non-Silicone Foundation

| | | %(w/w) |
|---|---|---|
| PHASE A | Skin conditioners | 17.0 |
| | Emulsifiers | 10.0 |
| | Viscosity enhancers | 9.0 |
| PHASE B | Sample VI | 6.0 |
| PHASE C | Dyes | 18.3 |
| PHASE D | Preservatives | 1.0 |
| PHASE E | Solvents | 35.0 |
| | Viscosity enhancers | 2.0 |
| | Chelating agents | 0.2 |
| PHASE F | Antioxidants | 0.5 |
| | Fragrances | 1.0 |

Summary table

| Sample/ | Raw material | P_{N, MAX}/ | Shear rate for | |
|---|---|---|---|---|
| Example | contained | Pa | F_{N, MAX}/s⁻¹ | T/° c. |
| SAMPLE I | — | 7385 | approx. 100 | 20 |
| SAMPLE II | — | 6621 | approx. 150 | 20 |
| SAMPLE III | — | 3183 | approx. 150 | 30 |
| SAMPLE IV | — | 7130 | approx. 100 | 60 |
| SAMPLE V | | 6950 | approx. 100 | 30 |
| SAMPLE VI | | 6503 | approx. 100 | 20 |
| EXAMPLE 1 | SAMPLE II | 229 | approx. 3 | 20 |
| EXAMPLE 2 | SAMPLE I | 331 | approx. 800 | 20 |
| | SAMPLE III | | | |
| EXAMPLE 3 | SAMPLE I | 92 | approx. 300 | 20 |
| EXAMPLE 4 | SAMPLE II | 61 | approx. 300 | 20 |
| EXAMPLE 5 | SAMPLE IV | 611 | approx. 800 | 60 |
| EXAMPLE 6 | SAMPLE V | 250 | approx. 3 | 20 |
| EXAMPLE 7 | SAMPLE VI | 3068 | approx. 800 | 20 |

1-21. (canceled)

22. A fluid cosmetic composition with a formulation comprising between 0.1 and 50% by weight of at least one fluid branched polymer obtained by reaction of the following components A, B and C:
A) linear polymer with a molecular weight Mw between 5000 Da and 50000 Da (expressed in equivalents of polystyrene), provided with reactive functional groups X in a chain at a concentration between 0.1 and 50 mmol/g of polymer;
B) linear polymer with a molecular weight Mw between 10000 Da and 100000 Da (expressed in equivalents of polystyrene), having terminal reactive functional groups of type Y capable of reacting with the functional group X in an addition or condensation reaction;
C) molecule o linear oligomer with a molecular weight between 50 Da and 1000 Da (or a mixture thereof) having a reactive functional group Y per molecule, capable of reacting with the functional group X in an addition or condensation reaction;
wherein said fluid branched polymer is a branched alkyl dimethicone in which:
A) is selected from polymethylhydrosiloxanes with the following structural formula: 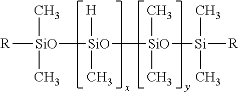
with x>2; y≥0; R═H, CH₃
B) is selected from linear polydimethylsiloxanes with terminal vinyl groups according to the following structural formula: 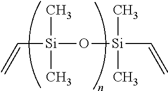
wherein n identifies a polymer with a molecular weight Mw between 10000 Da and 100000 Da;
C) is selected from alpha olefins of general formula CH₂═CH—R with R being an alkyl group containing from 6 to 50 carbon atoms;
wherein:
the molar ratio of the functional groups of B and A is between 0.01 and 0.5,
the molar ratio of the functional groups of C and A is between 0.5 and 1.2, and
the reactive group X of A is capable of reacting with the reactive sites Y of B and C;
and wherein the reaction is a simultaneous hydrosilylation reaction in the presence of a hydrosilylation catalyst.

23. The fluid cosmetic composition of claim 22, wherein the polymers A and B are selected from the families of polymers used in cosmetics, in particular silicones, polyesters, and polyurethanes.

24. The fluid cosmetic composition of claim 22, wherein the reactive functional groups X and Y are selected from complementary reactive groups, in particular Si—H, carboxyl, vinyl, hydroxyl, alkoxy (—O—CH₃, —O—CH₂CH₃, —O—CH(CH₃)₂) groups, amines, isocyanates.

25. A cosmetic product with the fluid cosmetic composition of claim 22.

26. The cosmetic product of claim 25, which is a lipstick.

27. The cosmetic product of claim 25, which is a foundation.

28. The cosmetic product of claim 25, which is a lip concealer.

29. The cosmetic product of claim 25, which is a primer.

30. A fluid branched polymer for a fluid cosmetic composition, wherein the branched polymer is the result of a reaction between the following components A, B and C:
A) linear polymer with a molecular weight Mw between 5000 Da and 50000 Da (expressed in equivalents of polystyrene), provided with reactive functional groups X in a chain at a concentration between 0.1 and 50 mmol/g of polymer;
B) linear polymer with a molecular weight Mw between 10000 Da and 100000 Da (expressed in equivalents of polystyrene), having terminal reactive functional groups of type Y capable of reacting with the functional group X in an addition or condensation reaction;
C) molecule o linear oligomer with a molecular weight between 50 Da and 1000 Da (or a mixture thereof) having a reactive functional group Y per molecule, capable of reacting with the functional group X in an addition or condensation reaction;
wherein said fluid branched polymer is a branched alkyl dimethicone in which:
A) is selected from polymethylhydrosiloxanes with the following structural formula: 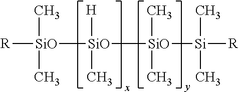
with x>2; y≥0; R═H, CH₃
B) is selected from linear polydimethylsiloxanes with terminal vinyl groups according to the following structural formula: 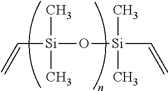
wherein n identifies a polymer with a molecular weight Mw between 10000 Da and 100000 Da;
C) is selected from alpha olefins of general formula CH₂═CH—R, with R being an alkyl group containing from 6 to 50 carbon atoms;
wherein
the molar ratio of the functional groups of B and A is between 0.01 and 0.5,
the molar ratio of the functional groups of C and A is between 0.5 and 1.2, and
the reactive group X of A is capable of reacting with the reactive sites Y of B and C;
and wherein the reaction is a simultaneous hydrosilylation reaction in the presence of a hydrosilylation catalyst.

31. The fluid branched polymer of claim 30, wherein the polymers A and B are selected from the families of polymers used in cosmetics, in particular silicones, polyesters, and polyurethanes.

32. The fluid branched polymer of claim 30, wherein the reactive functional groups X e Y are selected from complementary reactive groups, in particular Si—H, carboxyl, vinyl, hydroxyl, alkoxy (—O—CH₃, —O—CH₂CH₃, —O—CH(CH₃)₂) groups, amines, isocyanates.
